(19) United States
(12) Patent Application Publication
(10) Pub. No.: US 2006/0254915 A1
(43) Pub. Date: Nov. 16, 2006

(54) Microchip electrophoresis method and apparatus

(75) Inventors: Takeshi Hirokawa, Higashihiroshima-shi (JP); Akihiro Arai, Kyoto-shi (JP)

Correspondence Address: KANESAKA BERNER AND PARTNERS LLP, SUITE 300, 1700 DIAGONAL RD, ALEXANDRIA, VA, 22314-2848, US

(73) Assignees: SHIMADZU CORPORATION, Kyoto-shi (JP); HIROSHIMA UNIVERSITY, Higashihiroshima-shi (JP)

(21) Appl. No.: 11/433,544

(22) Filed: May 15, 2006

(30) Foreign Application Priority Data
May 13, 2005 (JP) 2005-141987

(51) Int. Cl. C07K 1/26; G01N 27/00

(52) U.S. Cl. 204/451; 204/601

(57) ABSTRACT

A microchip electrophoresis method performs sample preconcentration and separation, and includes a sample concentration step and a sample separation step. The sample concentration step performs sample concentration in a preprocess section of an electrophoresis channel by isotachophoresis after electrically suspending at least one branch channel branching from the electrophoresis channel. The sample separation step performs sample separation in a separation section following the preprocess section by zone electrophoresis or gel electrophoresis by introducing an electrolyte from the branch channel to the separation section by applying a predetermined voltage to the branch channel after the sample solution in the electrophoresis channel passes an intersection between the electrophoresis channel and the branch channel.

### BACKGROUND OF THE INVENTION AND RELATED ART STATEMENT

The present invention relates to a method and apparatus for microchip electrophoresis, more specifically a method and apparatus for microchip electrophoresis capable of performing on-line sample preconcentration.

Microchip electrophoresis is an analytical method that performs electrophoresis of a sample in a channel that is formed in a sheet-shaped microchip, and is capable of analyzing trace samples for proteins, nucleic acids or the like at high speed and with high resolution. For microchip electrophoresis, however, low concentration sensitivity due to the channel width and depth restrictions has been the most significant drawback. Such a fault can be improved by employing a highly sensitive detector, such as a laser-induced fluorescence and mass spectrometer. Using these specialized detectors, however, may place a limit on analyzable samples or reduce the convenience of the apparatus. Thus, there has been a demand for high-sensitivity analysis using a system furnished with a highly versatile UV detector.

Employing an offset structure for the branch channel at the sample injection section is a simple way to increase the amount of the sample to be introduced. However, this merely achieves a two- to three-time higher effect, and can reduce the resolution. Accordingly, incorporating some type of on-line preconcentration procedure becomes essential to enable the introduction of a large quantity of sample into the channel without sacrificing the resolution.

Conventionally, capillary electrophoresis, which utilizes a capillary tube, has employed the following on-line preconcentration techniques: (1) stacking, (2) electrokinetic injection, (3) transient isotachophoresis, and (4) electrokinetic supercharging (see, for example, patent reference 1 and non-patent reference 1).

Stacking is a technique to perform on-line sample preconcentration by introducing a diluted sample plug into an electrophoretic buffer (supporting electrolyte). It utilizes the potential difference between the sample plug and the electrophoretic buffer to concentrate the sample at the interface between the two. If the sample plug is too long, however, the resulting high potential gradient increases the overall electroosmotic flow. This reduces the separation window, thereby causing insufficient separation. In addition, the mismatched electroosmotic flow between the sample plug and the electrophoretic buffer causes band broadening.

Transient isotachophoresis achieves the concentration effect of isotachophoresis by voltage application when sample ions, together with paired ions, are interposed between an electrolyte containing strong acids as leading ions having a greater mobility than any ion in the sample (leading electrolyte: LE), and an electrolyte containing weak acids (or amino acids) as terminal ions having a lesser mobility than any ion in the sample (terminal electrolyte: TE), and by creating a transient isotachophoretic state by subsequently reintroducing a leading electrolyte, while, at the same time, causing a transition to a separation state by means of zone electrophoresis (or gel electrophoresis). This technique can control the aforementioned shortcomings of-the stacking method.

Electrokinetic supercharging is a combination of the aforementioned transient isotachophoresis and an electrokinetic injection technique suited for mass loading of a low-concentration sample. The method injects a sample into a leading electrolyte-loaded channel by electrokinetic injection, and isotachophoretically concentrates the sample after injecting a terminal electrolyte. This technique has the advantage of securing a sufficient separation window even after the transition is made from an isotachophoretic state to zone electrophoretic state, since large sample quantity can be introduced as a sharp zone.

Patent Reference 1: Japanese Laid-Open Patent Publication No. 2004-325191

Non-patent Reference 1: “Bunseki Kagaku” 2003, Vol. 52, No. 12, pp. 1069-1079

These various preconcentration techniques discussed above are adaptable to capillary electrophoresis without any problem, but an attempt to accomplish on-line preconcentration by means of these techniques in microchip- electrophoresis gives rise to various problems attributable to the apparatus's structural constraints.

For example, a situation will be explained below where sample preconcentration and separation are performed in a microchip having a single channel, which is not branched, as shown in FIG. 8(a), using the aforementioned electrokinetic supercharging technique. After loading the channel with a leading electrolyte (LE), a sample solution is electrokinetically injected from an injection port (port #3), and then a terminal electrolyte (TE) is injected from port #3. Since the sample solution is interposed between the leading electrolyte and the terminal electrolyte, a large quantity of the sample can be introduced into the channel without diffusion. By subsequently replacing the terminal electrolyte with a leading electrolyte at port #3 and applying voltage to the channel, separation of the sample elements is carried out by means of zone electrophoresis or gel electrophoresis.

This method, however, is cumbersome, as the solution in port #3 needs to be repeatedly replaced in the order of leading electrolyte, sample solution, terminal electrolyte, and supporting electrolyte. Another problem arises in the case where the leading electrolyte contains a high viscosity polymer as a separation medium, as it requires the preparation of a separate leading electrolyte, which contains no polymer, in order to circumvent the drawback attributable to entraining air bubbles when the terminal electrolyte (TE) is replaced with the leading electrolyte.

In the case of performing preconcentration and separation of a sample by means of the aforementioned electrokinetic supercharging using a microchip that has a cross channel configuration as illustrated in FIG. 8(b), it is advantageous to increase the voltage levels at ports #1 and #2 while the sample is injected from port #3 so that a sufficient voltage level is applied to the cross section to increase the effect of concentration by means of isotachophoresis. However, when the sample passes the cross section, the voltage applied to ports #1 and #2 needs to be lower than that applied to the cross section in order to prevent the concentrated sample from being drawn towards ports #1 and #2. Thus, voltage regulation becomes complicated.

It is therefore an object of the present invention to provide a method for microchip electrophoresis and the apparatus for the method capable of performing an on-line concentration and separation of a sample in a simplified manner.

Further objects and advantages of the invention will be apparent from the following description of the invention.

### SUMMARY OF THE INVENTION

The microchip electrophoresis method in the present invention devised for solving the aforementioned problems, is a microchip electrophoresis method for performing sample preconcentration and separation using a microchip that includes an electrophoresis channel comprising a preprocess section and a separation section formed within the sheet-shaped member, at least one branch channel branching out of said electrophoresis channel, and a plurality of ports comprising holes formed from one surface of said sheet-shaped member to the channels in the locations respectively corresponding to the ends of the channels.

The method comprises a sample concentration step whereby sample concentration is performed by means of isotachophoresis within said preprocess section after electrically suspending said branch channel, and a sample separation step whereby sample separation is performed by means of zone electrophoresis or gel electrophoresis by introducing an electrolyte from said branch channel to said separation section by applying a predetermined voltage level to the branch channel after the sample solution within said electrophoresis channel passes the intersection between said electrophoresis channel and said branch channel.

Moreover, it is desirable for the microchip electrophoresis method according to the present invention to comprise the aforementioned sample concentration step with a step whereby a leading electrolyte is loaded into said electrophoresis channel and said branch channel, a step whereby a sample solution is electrokinetically injected from the port provided in said electrophoresis channel in the preprocess section, a step whereby a terminal electrolyte is injected from the port provided in said electrophoresis channel in the preprocess section, and a step whereby isotachophoresis is performed by electrically suspending said branch channel while applying voltage to said electrophoresis channel.

The microchip electrophoresis apparatus according to the present invention is a microchip electrophoresis apparatus provided with a sample preconcentration function, comprising a microchip that includes an electrophoresis channel comprising a preprocess section and a separation section formed within the sheet-shaped member, at least one branch channel branching out of said electrophoresis channel,-and a plurality of ports comprising holes formed from one surface of said sheet-shaped member to the channels in the locations respectively corresponding to the ends of the channels; electrodes respectively disposed at the ports of said microchip; a power supply for applying a predetermined voltage level to each of said electrodes; a relay switch disposed between the electrode disposed at the branch channel of said microchip and the power supply connected to said electrode; and a control unit for controlling said power supply and relay switch so that said relay switch is turned off to place said branch channel in an electrically suspended state during sample concentration, and said relay switch is turned on to apply a predetermined voltage level to said branch channel once the sample solution passes the intersection between said electrophoresis channel and the branch channel.

It is desirable, but not limited, to employ for the “electrode disposed at each port” herein, an electrode comprised of a conductive thin film disposed on the microchip surface at the periphery of each port by means of vapor deposition or the like. A needle shaped electrode, which would be inserted into each port, may also be acceptable.

Moreover, the microchip used in the aforementioned method and apparatus for microchip electrophoresis desirably has the channel that is shaped so as to make several meandering U-turns in the preprocess section.

Conventional methods for microchip electrophoresis require simultaneous voltage control for all ports in each step, i.e., sample injection, concentration, and separation. In contrast, in accordance with the microchip electrophoresis method in the present invention, it is unnecessary to apply voltage to the branch channel when the sample passes through the intersection of the aforementioned electrophoresis channel and branch channel, reducing the cumbersomeness of the voltage regulation. Moreover, once the sample passes through the intersection, the electrolyte loaded within the branch channel flows into the electrophoresis channel. Thus, the method can omit the extra replacement step for port #3 into which the sample and terminal electrolyte have been injected.

As described above, in the case of utilizing a microchip having a preprocess section comprised of several meandering U-turned channel sections, a longer preprocess section can be formed in comparison to that in a linear channel construction within the same size chip. As a result, a larger sample quantity can be introduced while at the same time performing concentration to thereby further improve the detection sensitivity.

### BRIEF DESCRIPTION OF THE DRAWINGS

FIG. 1 is a schematic diagram showing the construction of the pertinent part of one example of the present invention's microchip electrophoresis apparatus.

FIGS. 2(a)-2(c) are diagrams that explain the channel construction of the microchip used in the example, and the steps of the electrophoresis method using the channels.

FIGS. 3(a) and 3(b) are electropherograms obtained by the method for tests 1 and 2, respectively.

FIG. 4 shows the current profile observed during test 1.

FIGS. 5(a) and 5(b) show the magnitude of sample concentration for tests 1 and 2, respectively.

FIG. 6 is a diagram of one example of microchip having a preprocess section in which the channel makes-several U-turns.

FIG. 7 is a CCD image of the U-turned section of the channel in the microchip during the preconcentration step.

FIGS. 8(a) and 8(b) are diagrams explaining a conventional microchip electrophoresis method using electrokinetic supercharging for microchips with a single channel and a cross channel, respectively.

### DETAILED DESCRIPTION OF PREFERRED EMBODIMENTS

The best mode of the invention will be explained below with reference to one example.

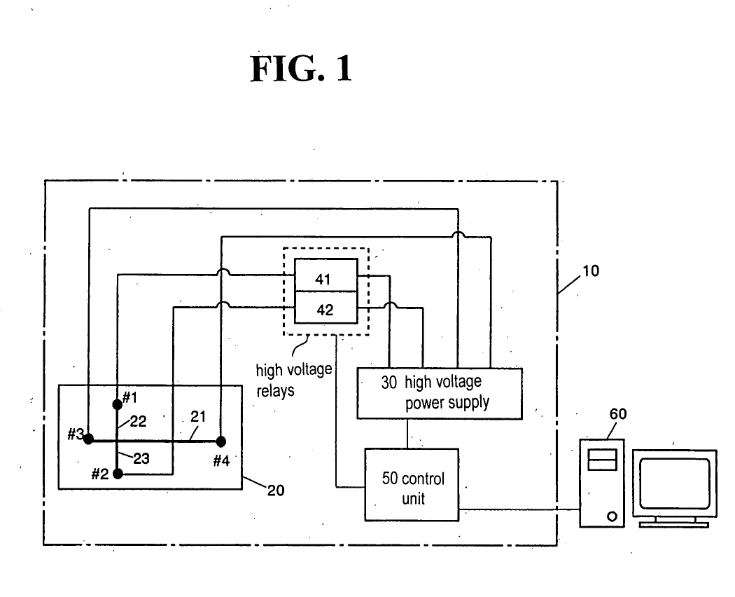

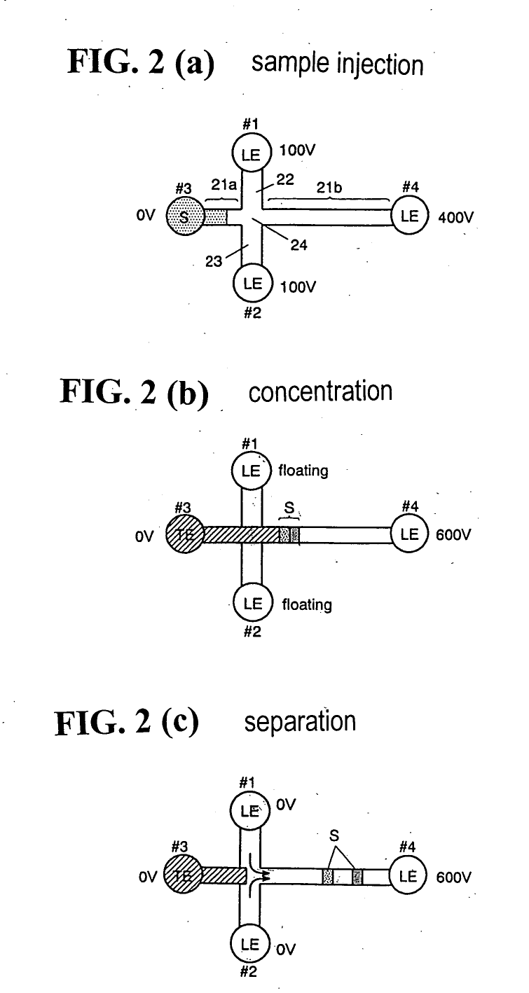

FIG. 1 shows the construction of the pertinent parts of the microchip electrophoresis apparatus according to the present invention, and FIG. 2 shows the electrophoresis steps using the apparatus.

The microchip 20 in the present invention has an electrophoresis channel 21, a cross channel comprised of two branch channels 22 and 23, which branch out from the electrophoresis channel 21, and ports #1-#4 disposed at the respective ends of the channels. The microchip 20 is comprised of a pair of transparent quartz plates or sheets; more specifically, the microchip 20 is prepared by pasting together a first sheet having groove-shaped channels formed on its surface and a second sheet having through holes formed at the locations corresponding to the respective ends of said grooves so that the grooves are positioned inside the microchip. The preconcentration of the sample takes place in the upstream side of the electrophoresis channel 21 from the intersection between the electrophoresis channel 21 and the branch channels 22 and 23 (referred to as branch section 24), and the separation of the sample takes place in the downstream side of the electrophoresis channel 21, and thus these regions are referred to as preprocess section 21a and separation section 21b, respectively, in the present invention.

Ports #3 and #4 are respectively located at the ends of the preprocess section 21a and the separation 21b of the electrophoresis channel 21, and ports #1 and #2 are respectively located at the ends of the branch channels 22 and 23. Around these ports #1-#4, electrodes made of conductive film (not show) are formed.

In the microchip electrophoresis apparatus 10 in this example, the aforementioned electrodes are connected to a high voltage power supply 30. Moreover, between the high voltage power supply 30 and each of the electrodes disposed at ports #1 and #2, high voltage relays 41 and 42 are disposed, respectively. The high voltage power supply 30 and high voltage relays 41 and 42.are controlled by a control unit 50. To the control unit 50, a personal computer 60 loaded with user specific software is connected. The analysis conditions set by the software are transmitted from the personal computer 60 to the control unit 50, while the resultant electrophoresis data is transmitted from the control unit 50 to the personal computer 60. Although omitted in the figures herein, the microchip electrophoresis apparatus 10 in this example is further provided with an autosampler for introducing a sample and electrolyte to port #3, a linear image UV detector for detecting the electrophoretic patterns for the range from the branch section 24 of the electrophoresis channel 21 to port #4, and the like.

One example of the electrophoresis method using the microchip electrophoresis apparatus described above will be explained below.

In the example, buffers satisfying the following conditions were used.

Buffer A (LE): 50 mM HCl-creatinine, 2% hydroxypropylmethyl cellulose (Mn=11,500), pH 4.8

Buffer B (TE): 10 mM capronic acid-creatinine, 2% hydroxypropylmethyl cellulose (Mn=11,500), pH 4.8

The compositions of sample solutions used in this example are as follows:

Test 1: 0.05 mM SPADNS (4,5-dihydroxy-3-(p-sulfophenylazo)-2,7-naphthalene disulfonic acid, trisodium salt) and Guinea Green B

Test 2: 0.05 mM SPADNS and Naphthol Green B

First, the electrophoresis channel 21 and branch channels 22 and 23 are fully preloaded with buffer A (LE), and the sample solution (S) is loaded into port #3. The sample is electrically injected for 20 seconds by applying 100V to ports #1 and #2, 0V to port #3, and 400V to port #4 (high voltage relays 41 and 42 are turned on) (FIG. 2(a)). Next, after replacing the contents in port #3 with buffer B (TE), the voltage is set at 0V and 600V for ports #3 and #4, respectively, and the high voltage relays 41 and 42 are turned off. Transient isotachophoresis is carried out, and the sample is concentrated in a sharp band without being affected by the branch channels 22 and 23 (FIG. 2(b)). Thirty seconds later, the high voltage relays 41 and 42 are turned on, and the voltage for ports #1-#3 is switched to 0V and port #4 to 600V. As a result, the buffer A (LE) preloaded in ports #1 and #2 is introduced into the separation section 21b behind the sample, and the separation of sample components by means of zone electrophoresis is carried out (FIG. 2(c)).

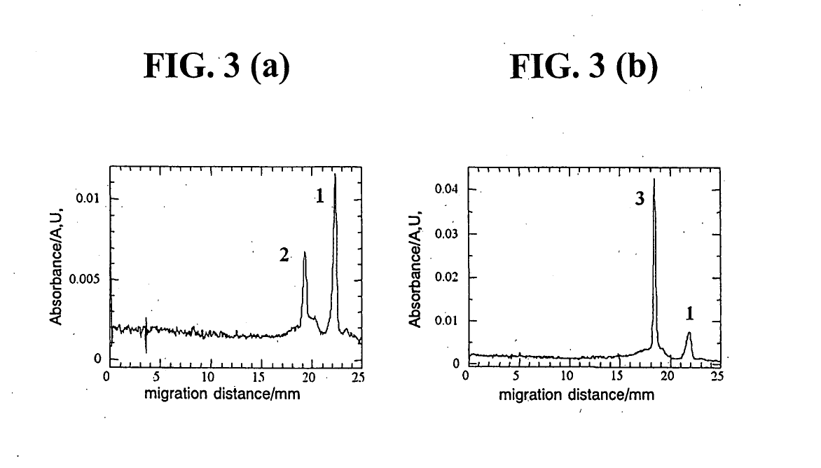

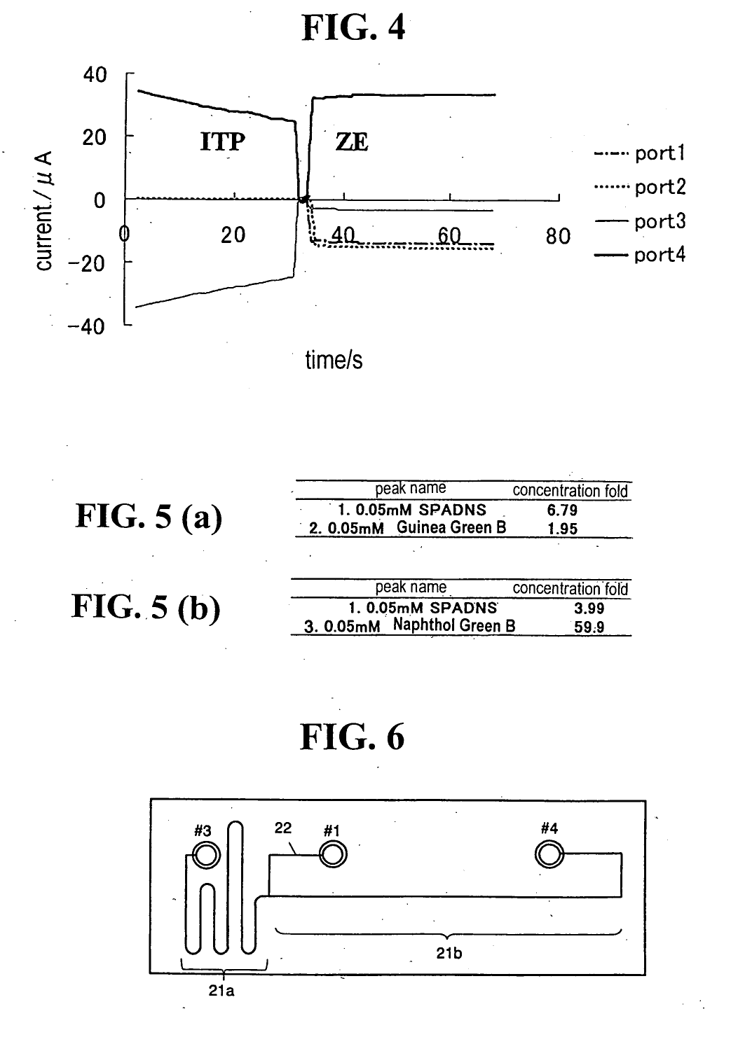

FIGS. 3(a) and 3(b) show the electropherograms and FIG. 4 shows the current profile observed during the above processes. As a result, the method for microchip electrophoresis used in this example achieved a maximum sample concentration of about 60-times, as compared to the general method for electrophoresis conducted as a comparative example which introduced the sample in a pinched manner from a branch channel for separation and detection (FIG. 5(b)). The multiple of sample concentration was obtained as follows. The absorbance before concentration was obtained by conversion from the molar absorption coefficient obtained by using a spectrophotometer and the groove depth of the microchip's separation channel; the values represent the ratios of absorbance after concentration to absorbance before concentration.

When a leading electrolyte used contains a high-viscosity polymer serving as a separation medium, as in the case of DNA size analysis, conventional methods require that the leading electrolyte be replaced with another leading electrolyte which contains no polymers (50 mM HCl-creatinine, pH 4.8 in the example) to prevent entrainment of air bubbles when the terminal electrolyte is replaced with the leading electrolyte at port #3. The method for microchip electrophoresis in the example, on the other hand, is structured so that the leading electrolyte preloaded in the branch channels 22 and 23 flows into the electrophoresis channel 21 once the sample passes the branch section 24. Thus, entrainment of air bubbles can be prevented, and the extra step of preparing a separate non-polymer leading electrolyte can be omitted.

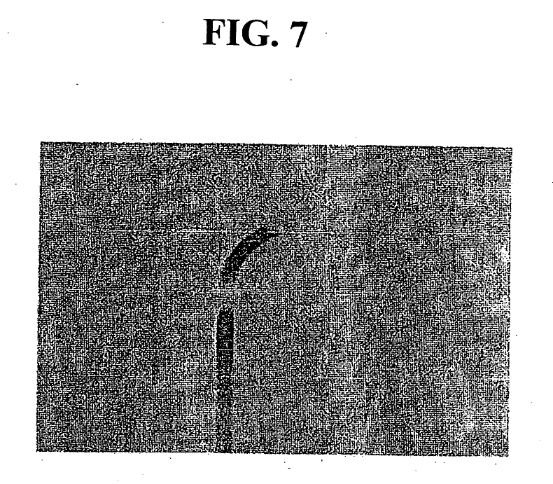

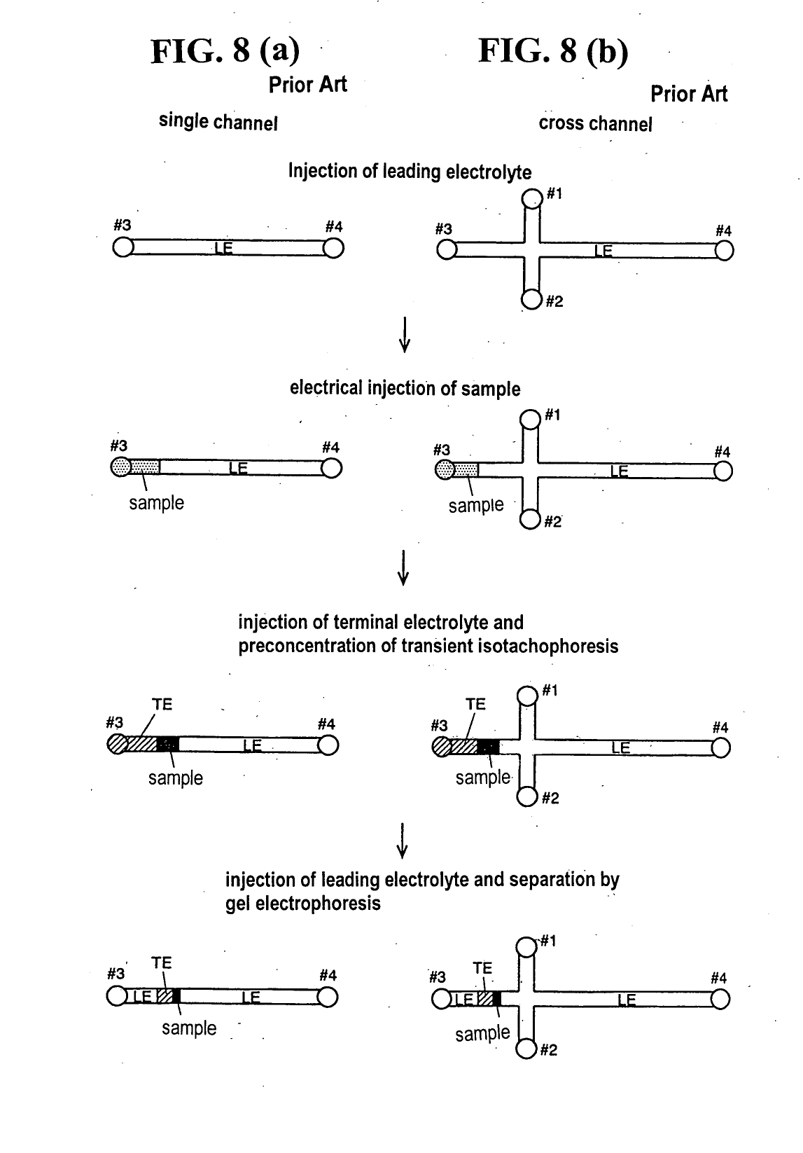

In the present invention, moreover, a microchip having a longer channel 21, which makes several meandering U-turns in the preprocess section 21a, may also be used, in addition to one that is comprised of a linear channel as shown in FIGS. 2(a)-2(c). FIG. 6 shows one example of such a channel structure. In this microchip, an electrophoresis channel 21 and one branch channel 22, which branches out from the electrophoresis channel 21, are formed, and the preprocess section 21a of the electrophoresis channel 21 is given a shape that makes five meandering U-turns. Usually, when applying voltage to such a meandering channel loaded with a uniform buffer, the sample zone tends to broaden in the longitudinal direction of the channel since the electric field lines are not uniformly distributed radially. When the sample is interposed between the leading electrolyte and the terminal electrolyte as in the case of the example, however, no sample zone broadening occurs even in the sections where the channel makes U-turns, as shown in the micrograph in FIG. 7. Thus, a large sample quantity can be introduced while concentrating the sample. In the case of performing preconcentration and separation of a sample using the microchip having such a channel structure in the same method as described above, 100-fold or greater concentration can theoretically be achieved.

The microchip electrophoresis apparatus and the method for electrophoresis using the apparatus according to the present invention have been explained above. The present invention, however, is not limited to the above example, and allows for various modifications within the scope thereof.

For example, the material used for the microchip in the present invention is not particularly limited, as long as it is microprocessable; in addition to the aforementioned quartz, Pyrex® glass, various types of ceramics, silicons, and resins such as PDMS (polydimethylsiloxane) may be used. Moreover, the microchip for use in the invention may be of one plate or sheet, in addition to one formed by pasting together two sheets as described in the above example. Such a microchip is produced by forming a fluidic channel within the single sheet, and forming a hole from one surface of the sheet through the channel in the location corresponding to the channel.

In the above example, moreover, the method for transient isotachophoresis using the most basic loading method, which interposes the sample solution between the leading electrolyte and the terminal electrolyte, has been explained. However, as described in the non-patent reference 1, various other loading methods are used in transient isotachophoresis, including one that adds reagents to a sample that act as leading and terminal ions. Various such loading methods are also applicable to the sample concentration step in the present invention's method for microchip electrophoresis.

In the above example, the voltage application to the branch channel is programmed to initiate after the sample passes the branch section by controlling the high voltage relay so as to turn on after performing isotachophoresis for a certain period of time following the injection of the terminal electrolyte. The present invention, however, is not limited to such a control method that utilizes a time program, and may also be configured to switch the high voltage relay by monitoring the absorbance at the branch section utilizing a point detection type UV detector disposed at the branch section, a linear imaging UV detector for detecting the electrophoretic pattern in the range between the branch channel and the end of the separation section, or the like, to determine when the sample passes the branch section.

The disclosure of Japanese Patent Application No. 2005-141987 filed on May 13, 2005 is incorporated herein as a reference.

What is claimed is:

1. A microchip electrophoresis method for performing sample preconcentration and separation, comprising:
a sample concentration step for performing sample concentration in a preprocess section of an electrophoresis channel by isotachophoresis after electrically suspending at least one branch channel branching from the electrophoresis channel, and
a sample separation step for performing sample separation in a separation section following the preprocess section of the electrophoresis channel by zone electrophoresis or gel electrophoresis by introducing an electrolyte from said at least one branch channel to said separation section by applying a predetermined voltage to the branch channel after the sample solution in said electrophoresis channel passes an intersection between said electrophoresis channel and said branch channel.

2. A microchip electrophoresis method according to claim 1, wherein said sample concentration step comprises:
a step of loading a leading electrolyte into said electrophoresis channel and said branch channel,
a step of injecting a sample solution electrokinetically from a port provided in the preprocess section of the electrophoresis channel,
a step of injecting a terminal electrolyte from the port provided in the preprocess section of the electrophoresis channel, and
a step of performing the isotachophoresis by electrically suspending said branch channel while applying voltage to said electrophoresis channel.

3. A microchip electrophoresis method according to claim 2, wherein said sample concentration step further comprises:
a first step of applying voltage at a first port for the at least one branch channel greater than that at a second port as said port provided in the preprocess section and less than that at a third port at the separation section after injecting the sample solution into the second part so that the sample solution is electrokinetically injected, and
a second step of applying voltage at the third port greater than that of the second port while no voltage is applied to the first port so that the isotachophoresis is performed.

4. A microchip electrophoresis method according to claim 3, wherein in the sample separation step, a voltage higher than that at the first and second ports is applied to the third port.

5. A microchip electrophoresis method according to claim 1, wherein a channel in said preprocess section of the microchip is shaped to have several meandering U-turns.

6. A microchip electrophoresis apparatus having a sample preconcentration function, comprising:
a microchip formed of a plate member, and including an electrophoresis channel having a preprocess section and a separation section formed in the plate member, at least one branch channel branching from the electrophoresis channel, and a plurality of ports comprising holes extending from one surface of the plate member to the channels at locations respectively corresponding to ends of the channels,
electrodes respectively disposed at the ports of said microchip,
a power supply for applying a predetermined voltage to each of said electrodes,
a relay switch disposed between the electrode disposed at the branch channel of said microchip and the power supply connected to said electrode, and
a control unit for controlling said power supply and said relay switch so that said relay switch is turned off to place said branch channel in an electrically suspended state during sample concentration, and said relay switch is turned on to apply a predetermined voltage to said branch channel once the sample solution passes the intersection between said electrophoresis channel and the branch channel.

7. A microchip electrophoresis apparatus according to claim 6, wherein the channel in said preprocess section of the microchip has several meandering U-turns.
